(19) United States
(12) Patent Application Publication
(10) Pub. No.: US 2003/0212313 A1
(43) Pub. Date: Nov. 13, 2003

(54) Method of diagnosing migraine

(75) Inventor: Wilma Marcia Harrison, Harrison, NY (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/393,486

(22) Filed: Mar. 20, 2003

Related U.S. Application Data
(60) Provisional application No. 60/378,496, filed on May 8, 2002.

(51) Int. Cl. A61B 5/00

(52) U.S. Cl. 600/300

(57) ABSTRACT

This invention relates to a novel improved three-item screener which is a valid and reliable screening instrument for migraine headaches. Its ease of use and operating characteristics suggest that it could significantly improve migraine recognition in primary care.

This application is based on U.S. Provisional Patent Application Serial No. 60/378,496, filed May 8, 2002.

### BACKGROUND OF THE INVENTION

This invention relates to a new, simplified method of diagnosing migraine.

Recent epidemiological studies show that migraine is an extremely common disorder with a one-year period prevalence in the United States of approximately 18% for women, and 6% for men. (Scher A I, et al., in: Crombie I K, ed. The epidemiology of pain. Seattle: International Association for the Study of Pain Press, 1999:159-170; Rasmussen B K, Cephalalgia 2001, 21:774-777; Lipton R B, et al., Headache. 2001, 41:646-657; Rasmussen B K, Cephalalgia 1995;15:45-68). In these studies, prevalence peaks in the early forties, with a female predominance of 3:1. The mean frequency of migraine headaches has been estimated to be approximately 1-2 per month, with headache severity characterized as “severe” or “very severe” by 60-80% of migraineurs. (Scher, et al.; Rasmussen, Cephalalgia 1995;15:45-68; Lipton, et al., Headache. 2001, 41:646-657)

The impact of migraine illness on health-related quality of life (HR-QoL) and on functional status is substantial, and includes impairment in work, social activities, and family life. (Stewart A L, et al., JAMA, 1989 Aug. 18, 262(7):907-13; Hu X H, et al., Arch Intern Med. 1999, 159:813-818; Osterhaus J T, et al., Pharmacoeconomics, 1992, 2:67-76; Murray C J, Lopez A D, Lancet 1997, 349:1498-1504; Lipton R B, et al., Migraine diagnosis and treatment: results from the American migraine study II. (in press)). During a typical migraine, individuals report that the attack results in severe functional impairment and/or complete bed rest in approximately 50% of attacks. (Lipton R B, et al., Headache 2001, 41:646-657) The impairment and impact on HR-QoL of migraine has been found to be equivalent or greater than for other chronic medical illnesses such as angina, diabetes and hypertension. (Scher A I, et al.)

Despite the frequency, severity, and negative economic, functional and HR-QoL impact of migraine, recent surveys suggest that less than half of current migraineurs have ever received a medical diagnosis of migraine. (Scher A I, et al.) The low level of appropriate diagnosis appears to be due, in part, to a low consultation rate. Even though healthcare utilization is increased among migraineurs, less than half of migraine sufferers have consulted a doctor with a complaint of headache in the previous year. The overwhelming majority of patients who seek care for headache do in primary care settings. (Clouse J C, Osterhaus J T, Ann Pharmacother 1994, 28:659-664) Another factor contributing to the low rate of migraine diagnosis may be the extremely short duration of a typical physician-patient contact in this setting (averaging approximately 5-8 minutes). (Scher A I, et al.) In a brief encounter, headaches may be treated as a relatively low medical priority. In addition poor physician-patient communication regarding an event that is not ongoing during the visit may further reduce diagnostic recognition of migraine. (Scher A I, et al.; Rasmussen B K, et al., Cephalalgia 2001, 21:774-777) Even under ideal circumstances appropriate diagnosis may be difficult. Any individual International Headache Society (IHS) criterion symptom of migraine, such as nausea, photophobia or phonophobia, may be absent in up to 40% of migraine sufferers. (Stewart W F, et al., Cephalalgia 1999, 19:107-114) Physicians may rely on aura for diagnosis, a feature that is absent in more than two-thirds of migraine sufferers. (Stewart A L, et al., JAMA, 1989 Aug. 18, 262(7), 907-13; Leone M, et al., Cephalalgia 1994, 14:280-284) Conversely, some symptoms of migraine may be present in patients who suffer from headaches that do not meet IHS criteria for migraine. (Scher A I, et al.) In addition, lack of operationalized descriptors for many migraine symptoms makes it difficult to determine which symptoms meet threshold levels that would qualify them as IHS criteria. Finally, many migraine sufferers have more than one headache type (e.g., tension headaches), complicating the diagnosis. (Lipton R B, et al., Headache 2001, 41:646-657; Rasmussen B K, et al., Cephalalgia 2001, 21:774-777)

One approach to improving diagnosis in primary care settings, recommended by the recent US Headache Consortium Guidelines, is to employ screening or case-finding instruments. (Scher A I, et al.) Though screening sometimes refers to detection of an illness early in its course, herein the inventors use the term to refer to the detection of previously undiagnosed individuals with migraine. An ideal screening instrument for migraine should be brief and easy to use (focus groups had previously suggested that three items was the optimal length). It should have operating characteristics that would make it useful in the primary care setting. Specifically, it should have sufficient sensitivity to detect patients in need of treatment, and sufficient specificity and positive predictive value to ensure that limited physician time is optimized.

There have been previous efforts to develop screening instruments for migraine. (Scher A I, et al.; Rasmussen B K et al., Cephalalgia 2001, 21:774-777; Lipton R B, et al., Headache 2001, 41:646-657; Rasmussen B K, et al., Cephalalgia 1995, 15:45-68) Some studies were conducted in specialty referral settings, or in the general population, and results may not be applicable to the setting of intended use, primary care. Other studies lacked “gold standard” confirmation of the migraine diagnosis, ie, verification by a headache specialist applying IHS criteria. Still others developed instruments that either had unfavorable performance characteristics or were too time-consuming to be useful in primary care.

Therefore, it was desirable to establish the validity and reliability of a brief, patient self-report screening instrument whose sensitivity and specificity would make it useful in the outpatient primary care setting. Since the setting of intended use was primary care, The inventors developed a validation procedure in which screening occurred in the primary care setting, and the gold standard diagnostic assessment was subsequently performed by headache experts. The inventors conducted a validation study using a 9-item screener, including test-retest reliability. The inventors then conducted a second study to compare the reliability of responses on the three item screener to the full 9 item scale.

### SUMMARY OF THE INVENTION

This invention accordingly relates to a method of diagnosing migraine in a subject validly and reliably which comprises (a) determining the degree of disability of said subject; (b) determining whether said subject is suffering from nausea; and, (c) determining whether said subject is suffering from sensitivity to light.

In a particular embodiment, the invention provides a method as described herein method, wherein determining said points (a), (b) and (c) comprises asking said subject exactly three questions addressing points (a), (b) and (c), respectively.

In another particular embodiment, the invention provides a method as described herein, wherein said method has a predictive value of greater than 90 percent.

In another embodiment, the invention provides a method as described herein, wherein said method may be self-administered by said subject or administered by a medical professional.

### BRIEF DESCRIPTION OF THE DRAWINGS

FIG. 1 shows subject disposition during testing of the method in accordance with the present invention.

FIG. 2 shows an ROC curve for the 9-item screener.

FIG. 3 shows adjusted odds ratios for individual migraine screener items: likelihood of “gold standard” diagnosis of migraine (items numbers being referenced to Table 1).

FIG. 4 shows an ROC curve for the three-item (nausea, photophobia and disability) ID MIGRAINE™ screener in accordance with the present invention.

### DETAILED DESCRIPTION OF THE INVENTION

Subjects and Methods

The validity and reliability studies were conducted in the United States at 27 Primary Care Practice (PCP) sites that were located in close proximity to one of 12 practice sites staffed by a headache expert, either a neurologist or internist who specialized in the evaluation and treatment of headaches. Headache expert sites were selected for their geographic diversity, and for the availability of a diagnostician with expertise in headache. Patients were enrolled between April and August of 2000.

All male or female subjects aged 18 to 55 years old, inclusive, who were visiting a PCP office for any medical complaint were qualified to participate in the initial screening phase of the study, if they were able to read and write English, had not participated in a previous Pfizer-sponsored migraine study, and if they reported 2 or more headaches in the previous 3 months. In addition, eligible subjects reported that their headaches limited their ability to work, study, or enjoy life; or reported that they wished to speak with a healthcare professional about their headaches. After one-third of the sample had been enrolled, the inventors began to exclude patients with a previous diagnosis of migraine in an attempt to ensure a higher proportion of non-migraineurs in the sample.

The study protocol was reviewed by a central IRB or, if available, by the local IRB at the individual site. The benefits and risks of study participation were explained to each patient, and written, informed consent was obtained. Patients were reassured that declining to participate would in no way affect the care or treatment that they had come to receive from their PCP.

Study Procedures

The validation study was divided into 2 phases: 1) a PCP-based migraine screening phase, and 2) a headache expert diagnostic evaluation phase. In the first phase, outpatients visiting their PCP office who met the entry criteria itemized above were asked to complete the migraine screener, a self-rated paper-and-pencil questionnaire.

The screener focused on nine questions. Eight questions were rated by the patient on an ordinal severity scale whose four descriptors were “never,” “rarely,” “less than half the time,” and “half the time or more.” These response options had been previously shown to improve the diagnostic accuracy of screening for migraine, with “rarely” categorized as a negative response with “never”, thus reducing false positive symptom reporting. (Terwindt G M, et al., Neurology 2000, 55:624-629) Four of these questions focused on the IHS-defined pain features of migraine and three on the IHS-defined associated symptoms of migraine. One question on aura was included. The ninth question focused on the number of days the patient said the headaches limited their ability to work, study or do what they needed to do. This question quantified the days of disability caused by a patient's headache over the previous three months. The objective was to identify the items with the greatest discriminative validity for migraine, and to develop the simplest possible instrument with the optimal operating characteristics. Additional questions, not used for case-finding, obtained information on age, gender, race, previous diagnosis and frequency of headaches.

In the validation study, the patient-based screener was not scored or discussed with the patient, but was reviewed for completeness by the PCP or a member of his or her staff. The patient was then referred to a headache specialist for structured diagnostic headache evaluation, within 2 weeks of PCP screening. Results of the migraine screener were not available to the headache specialist.

The headache specialist evaluation included a medical history, physical examination, comprehensive neurological history and examination (including additional diagnostic tests if clinically indicated); and a semi-structured interview that included the IHS features of migraine supplemented by additional questions relating to family history, and medical treatment history. The headache expert was encouraged to probe for clinical information necessary to clarify the differential diagnosis.

Based on the evaluation detailed above, the headache specialist was asked to provide an IHS checklist (Stewart W F, et al., Cephalalgia 1999, 19:107-114) as well as a clinical diagnosis of migraine (IHS 1.1, 1.2), migrainous disorder (IHS 1.7), or other headache. The a priori “gold standard” diagnosis of migraine against which the screener performance was evaluated was operationally defined as any patient who received a migraine diagnosis based on either of the two methods, clinical or IHS diagnosis.

Health-Related Quality of Life and Functional Measures

Patients completed several self-administered HR-QoL and functional measures, including the Migraine-Specific Quality-of-Life Questionnaire, version 2.1 (MSQ) (Rasmussen B K, Cephalalgia 2001, 21, 774-777), a 14-item scale designed to assess the effect of migraine on health-related quality of life. Patients also completed the Migraine Disability Assessment (MIDAS) questionnaire (Stewart W F, et al., Pain 2000, 88:41-52), which asks patients to score their level of headache-related disability in five domains, including days of activity limitation in work, chores (household work), and non-work activities (social, family and leisure activities). In addition, patients completed the Migraine-Related Work Productivity Questionnaire (WPQ-24) (Rasmussen B K., Cephalalgia 2001, 21, 774-777), a 24-item scale designed to assess the effect of migraine on work productivity and work functioning. Finally, they completed the Henry Ford Hospital Headache Disability Inventory (HDI) (Rasmussen B K., Cephalalgia 2001, 21:774-777; Lipton R B, et al., Headache. 2001, 41:646-657), a 25-item scale designed to assess the effect of migraine on daily activities and functioning.

To assess test-retest reliability, a subset of approximately 25% of patients at each PCP site were randomly selected to complete a second migraine screener at home, approximately 2-7 days after completing the first screener at the PCP office, but prior to visiting the headache specialist. Participants in the reliability study were asked to mail the second screener directly to an independent study monitor to minimize the possibility that the headache expert might see it.

The original migraine screener was a 9-item scale, with a choice of four responses permitted for the first 8 items, and a numerical response required for the ninth item on disability. After analysis of the initial data set, a 3-item screener emerged with the optimal balance of operating characteristics and brevity. The inventors were concerned that the 3 items, embedded in a longer questionnaire, might perform differently than the 3 items as a stand-alone scale. The inventors therefore conducted an additional study at 9 PCP sites from the previous study using a new patient sample. The goal was to assess the level of agreement between an abbreviated, 3-item version used in isolation vs. the 3 items embedded in the original screener. As a secondary aim, the inventors also compared the original 4 category response options with a binary (yes/no) response option in an attempt to further simplify the instrument. Patients were recruited in the PCP setting using the same entry criteria, and were administered, in random order and at 5±2 day intervals, 3 versions of the screener: 1) the 9-item screener with ordinal responses, 2) the 3-item screener with ordinal responses, and 3) the 3-item Identification of (ID) Migraine™ Screener with binary responses.

Statistical Analyses

Descriptive statistics were performed on the demographic and clinical variables, as well as on responses on the migraine screener.

Item Selection and Validity Assessment

Each of the 9 migraine screener items was analyzed for item-total correlations and the influence of item elimination on internal consistency (Cronbach's alpha). The inventors planned to eliminate items with poor (<0.20) item-total correlations. The inventors further planned to exclude highly correlated items. A combination of statistical analysis and clinical/medical judgment decided if any questions are redundant and which questions to potentially delete.

Validity Assessment

Sensitivity and specificity of individual items were evaluated against the headache specialist's diagnosis. The inventors planned to treat the response to each item as a binary variable with a “no” assigned to responses of “never” or “rarely” and “yes” assigned to responses of “less than half the time” or “half the time or more.” The inventors also performed exploratory analyses to examine how varying the cut-score for “yes-no” assignment changed the level of agreement between individual items on the migraine screener and the equivalent physician-rated items.

To identify the optimal migraine screener, the following analyses were performed.

First, using the total score of the 9-item migraine screener against the gold standard, the receiver-operator characteristic (ROC) curve was generated. Using the method proposed by Halpern (Rasmussen B K, Cephalalgia 2001, 21:774-777), the optimal operating point (OOP) was determined from the ROC curve. The OOP serves as a cut-score for classifying subjects as likely migraine suffers vs. non-migraine sufferers. Based upon the OOP, sensitivity and specificity and their 95% two-sided confidence intervals were calculated.

Second, sensitivity and specificity were calculated using selected migraine screener items based on an algorithm that paralleled the operationalized IHS criteria for migraine.

Third, a logistic regression analysis was performed with the gold standard diagnosis as the dependent variable, and individual screener items as predictor variables to determine which combination of items was independently associated with the gold standard diagnosis of migraine. Based on the results of the logistic regression, empirical combinations were chosen using the 3 items with the highest individual sensitivities and specificities. An optimal operating point was then determined from the ROC curve for short versions of the scale in an attempt to identify the most efficient diagnostic screener.

Test-Retest Reliability Assessment

The reliability assessment was performed using the test-retest sample. The test-retest reliability of the refined total score for the migraine screener was evaluated with an intra-class correlation coefficient using the Shrout and Fleiss' formula. (Rasmussen B K, Cephalalgia 2001;21 :774-777) Using the intra-class correlation coefficient, the screener refined total scores at time 1 (initial screening visit) was compared to the time 2 (re-test visit) scores.

The kappa statistic and percent agreement were calculated to compare the responses to each of the nine items as binary variables with a “no” assigned to responses of “never” or “rarely” and “yes” assigned to responses of “less than half the time” or “half the time or more”, at time 1 with time 2.

The optimal migraine screener served as a cut-score for classifying subjects as likely migraine sufferers vs. non-migraine sufferers. Test-retest reliability was also calculated on this binary scoring of the migraine screening questionnaire. For this purpose, the kappa statistic was used to compare Migraine diagnosis by the optimal migraine screener at time 1 with time 2.

The adequacy of the kappa coefficient (K) was evaluated using the following descriptive ranges by which a kappa of 0.40-0.59 is considered to be “moderate,” a kappa of 0.60-0.79 is considered to be “substantial,” and a kappa>0.80 is considered to be excellent agreement. (Rasmussen B K, Cephalalgia 2001, 21:774-777)

Secondary analyses examined the impact of the following variables on the sensitivity and specificity of the optimal migraine screener: sex, age, history of a previous diagnosis of migraine headache, and presence or absence of other headache comorbidity.

Results

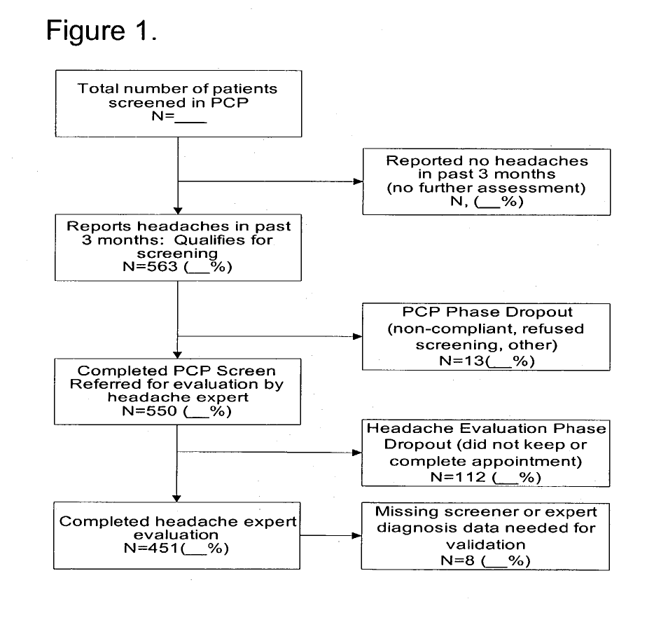

The inception cohort consisted of 563 patients who met study eligibility criteria and agreed to participate. Of the 563 patients who were screened and referred for evaluation by a headache expert, 451 (80%) completed the structured diagnostic assessment and constitute the validation sample (FIG. 1). The validation sample had a mean age of 39.3±10.1 years, and consisted of 341 females (75.6%). Three hundred twelve patients (69%) were white, 105 (23%) were black, 13 (2.9%) were Asian, and 21 (4.7%) reported other races; 223 (50%) were currently married, 143 (32%) had never married, 70 (15%) were divorced, separated, or widowed, and 15 patients had uncertain marital status. In terms of current work status, 332 (74%) reported being fully employed, 49 (11%) reported part-time employment, 30 (7%) reported being homemakers, 22 (5%) reported being unemployed, 13 (3%) reported being students, and 3 (1%) reported being retired. Among patients who eventually received a “gold standard” diagnosis of migraine, 124 (28%) reported having received a previous migraine diagnosis.

The 99 patients who completed the screener and were referred, but did not complete, the diagnostic evaluation by a headache expert showed only modest differences from the validation sample: they tended to be younger (mean±sd, 31.0±11.6 years vs. 39.3±10.1 years), reported less severe headache pain (moderate-to-severe, 82.7% vs. 92.0%, p<0.01), and reported less associated nausea (42.7% vs. 52.5%; p=0.07). These differences are consistent with previously reported differences among patients seeking vs. not seeking medical help for headaches. (Stewart W F, et al., Cephalalgia 1999, 19:107-114) Headache experts assigned a diagnosis based on both a clinical evaluation, and based on IHS criteria (using a semi-structured interview). The agreement between diagnostic methods was very high: 97.6% of patients receiving a clinical diagnosis of migraine were also diagnosed with migraine based on IHS criteria. Similarly, 92.2% of patients who met IHS criteria for migraine were also given a clinical diagnosis of migraine. An additional 8 patients received a clinical diagnosis of migrainous disorder, though none of these patients received an IHS criteria-based diagnosis of either migrainous disorder or migraine. A consensus decision was reached, prior to performing the validation analysis, that these 8 patients would not be classified as having a gold standard diagnosis of migraine.

Sensitivity and Specificity of Individual ID Migraine™ Screener Items

The inventors calculated the sensitivity and specificity of each of the 9 individual items on the long version of the migraine screener. The results show that the individual items have sensitivities ranging from a low of 43% for aura to a high of 94% for pain intensity (Table 1). Specificity was highest for nausea (81%), photophobia (74%), and aura (74%). Items providing levels of sensitivity and specificity greater than 50% each include one-sided pain, exacerbation with activity, nausea, photophobia, phonophobia, and functional impairment.

Exploratory analyses examined how varying the cut-score for “yes-no” assignment changed the level of agreement between individual items on the migraine screener and the equivalent physician-rated items. Consistent with expectations (Rasmussen B K, Cephalalgia 2001, 21:774-777), the inventors found that the “never/rarely” vs. “less/more-than-half-the-time” dichotomization yielded the highest kappa's, in the range of 0.23 to 0.47. These kappa's for individual items are fair, supporting the plan to combine items.

Identifying the Optimal Migraine Screener

The inventors employed three different approaches to identify the optimal migraine screener. The first approach involved selecting migraine screener items based on an algorithm that paralleled the operationalized IHS criteria for migraine. The results of this item definition yielded a screener with a sensitivity of 0.81 (95% Cl, 0.77-0.85) and a specificity of 0.69 (95% Cl, 0.58-0.79).

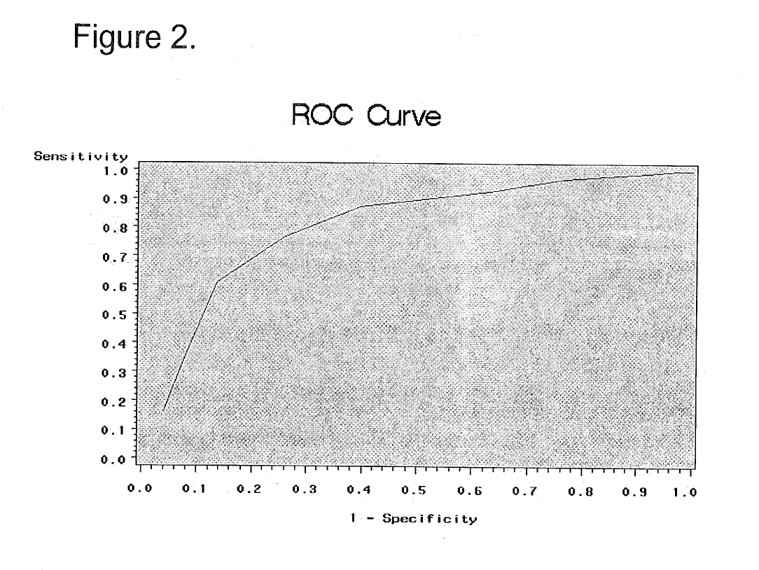

The second method involved simply summing the binary responses to the 9 screener items. The inventors generated an ROC curve (FIG. 2) by plotting the sensitivity of the screener total score against one minus the specificity (Rasmussen B K, Cephalalgia 2001, 21:774-777). The optimal operating point was determined to be a total score of 6, which yielded a sensitivity of 0.77 (95% Cl, 0.72, 0.81) and a specificity of 0.74 (95% Cl, 0.62, 0.84). Relative to the application of the IHS algorithm, this empirical approach resulted in a small decrement in sensitivity, but a small increment in specificity.

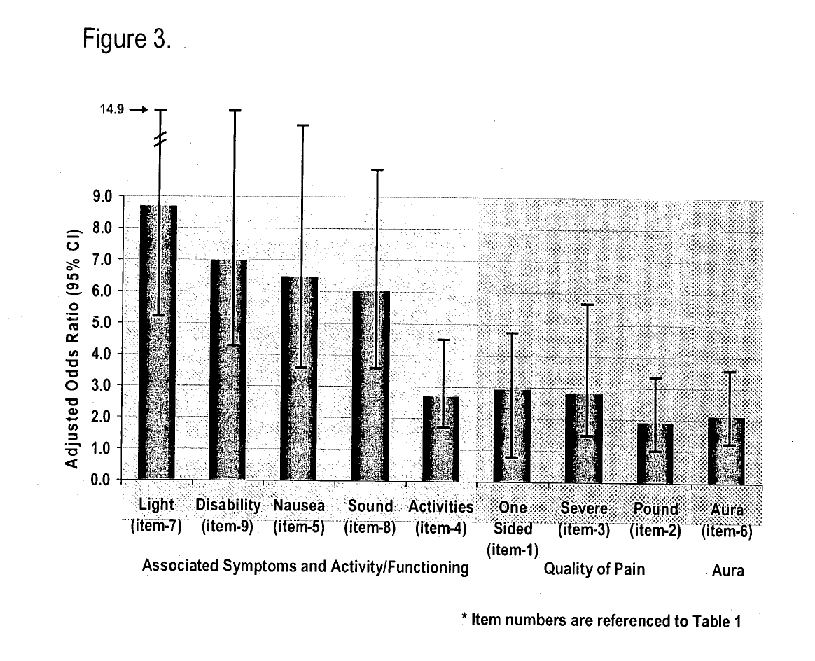

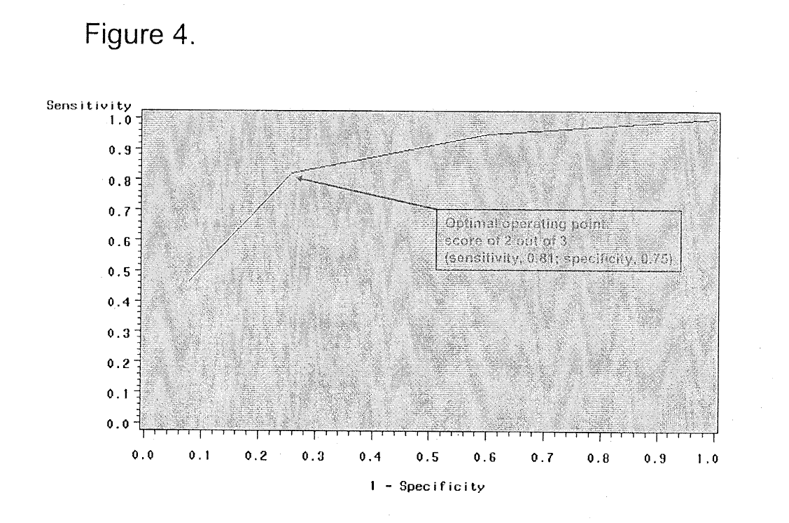

The third approach used a logistic regression analysis to determine which combination of items was independently associated with the gold standard diagnosis of migraine (FIG. 3). In univariate models each item was found to be significantly associated with migraine status. When all 9 items were forced into the model, 3 variables had a strong and significant association with the diagnosis of migraine: nausea, photophobia, and disability (any day in the previous 3 months). Other IHS criteria did not yield significant additional predictive value when included in models containing these 3 variables. Empirical testing of combinations of these 3 items found that the optimal total score was 2 (ie, scored positive on any 2 out of the 3 items). This yielded a sensitivity of 0.81 (95% Cl, 0.77-0.85) and a specificity of 0.75 (95% Cl, 0.64-0.84). The ROC curve for the 3-item screener is shown in FIG. 4. In the primary care setting, the 3-item ID Migraine™ screener was found to have a positive predictive value of 93.3.

The reliability of both the long (9-item) and short (3-item) versions of the migraine screener were evaluated. The internal consistency of the items in the long-form screener (binary scoring) was found to be good, with a Cronbach's alpha for the total scale of 0.70. Deleting any single item attenuated the alpha, suggesting that all items contributed to internal consistency. The item-to-total score correlations ranged from 0.27 to 0.57, suggesting that no single item showed too high a correlation.

For the 123 patients who completed the migraine screener a second time, test-retest reliability was found to be good. For the 3-item ID Migraine™ Screener (binary response) the test-retest kappa's for individual items ranged from 0.61 to 0.69. For the total score on the 3-item ID Migraine™ Screener (binary response), the kappa was 0.68 (95% Cl, 0.54-0.82). The test-retest interval (0-2 days vs. 3-5 days vs. 6 or more days) was found to have no influence on agreement.

The inventors examined the performance of the 3 items in subgroups defined by age, gender, previous diagnostic status, and headache co-morbidity. There were only modest differences in the performance of the ID Migraine™ Screener in these clinically relevant subgroups (Table 2). Most notably, the screener appeared to have somewhat lower sensitivity in males compared to females (0.65 vs. 0.86), but higher specificity.

An additional study was conducted in 9 PCP sites to confirm the agreement between the original 9-item long form of the migraine screener and the 3-item ID Migraine™ Screener. In a randomly assigned order, 161 patients received each of the migraine screeners at approximately 5-day intervals. Because of an a priori decision to exclude any patient who fell outside a 14-day test-retest window, data on 134 patients were included in the analysis. The results of the analysis found a 90.2% agreement between the 9-item long form and the 3-item screener, yielding a kappa of 0.73 (95% Cl: 0.57, 0.89).

Discussion

The current study demonstrates that the 3-item ID Migraine™ Screener, consisting of questions on disability, nausea and photophobia, is a valid and reliable screening instrument for migraine headaches in the primary care setting. Its use in the PCP setting was associated with a sensitivity of 0.81 (95% Cl, 0.77-0.85) and a specificity of 0.75 (95% Cl, 0.64-0.84), relative to a migraine diagnosis assigned by an expert.

No additional gain in sensitivity or specificity was achieved by use of the longer 9-item version of the screener, whether using a quantitative scale, or the application of an algorithm based on IHS criteria. Use of a cut score of 6 (the optimal operating point for the 9-item screener as determined by an ROC analysis) yielded a sensitivity of 0.77 (95% Cl, 0.72, 0.81) and a specificity of 0.74 (95% Cl, 0.62, 0.84). The IHS scoring approach yielded a somewhat higher sensitivity of 0.81 (95% Cl, 0.77-0.85) but a somewhat lower specificity of 0.69 (95% Cl, 0.58-0.79). Neither of these results was an improvement over the 3-item ID Migraine™ screener.

The presence of aura is the defining feature for one of the main clinical subtypes of migraine, comprising up to one-third of cases. (Lipton R B, et al., Headache. 2001, 41:646-657; Stewart A L, et al., JAMA, 1989 Aug. 18, 262(7), 907-13) Physicians frequently rely on aura as a cardinal symptom of migraine, as suggested by the 1.9-fold higher rate of medical diagnosis of migraineurs with aura compared to those without aura. (Stewart A L, et al., JAMA, 1989 August 18, 262(7):907-13; Leone M, et al., Cephalalgia 1994, 14:280-284) In the current validation study aura had good specificity but relatively low sensitivity (43%). Though aura is significantly associated with migraine status in univariate analyses, a logistic regression analysis found that aura did not add significantly to the performance of the 3-item screener. This occurs because most patients who have migraine with aura also meet criteria for migraine without aura, and specifically have at least two out of the three migraine screener items, disability, nausea, and photophobia. (Rasmussen B K, Cephalalgia 2001, 21:774-777)

The performance of the 3-item ID Migraine™ screener is approximately equivalent to the performance of the Patient Health Questionnaire, which is the patient-rated version of the depression section of the PRIME-MD® (sensitivity, 85%; specificity, 75%). (Rasmussen B K, Cephalalgia 2001, 21:774-777) Similarly, the ID Migraine™ Screener performs with a sensitivity and specificity in the same range as other commonly utilized medical screening tests such as the PSA for prostate cancer (sens, 75%; spec, 74%), the Pap test for cervical cancer (sens, 50-70%; spec, 92-95%) and the ECG for MI (sens, 50-60%; spec, 95%). (Lipton R B, et al., Headache, 2001, 41:646-657)

The difficulty in validating a migraine screening instrument with higher sensitivity and specificity than obtained in the current study may be attributable, in part, to the nature of migraine as an episodic illness with symptoms that vary from attack to attack within, and among, individuals. Appropriate diagnosis relies on retrospective assessment of symptoms that are rarely present at the time of evaluation, and for which there are no physical manifestations or confirmatory laboratory tests. One study shows that among neurologists asked to assign a headache diagnosis based on review of patient videotapes, kappa's ranged from 0.55 to 0.81. (Lipton R B, et al., Headache, 2001, 41:646-657) If this rate of inherent diagnostic uncertainty is taken as a ceiling, then the performance of the 3-item ID Migraine™ Screener may be quite close to a theoretical performance limit. Diagnosis of migraine in the primary care setting is reduced by the same variables.

In light of the difficulty inherent in screening and diagnosing migraine based on retrospective symptom reports, it is interesting to note that the performance of the screener was optimized by including a disability item as one of the 3 screener questions. Inclusion of this item distinguishes the ID Migraine™ Screener from previous symptom-based migraine screeners with lower sensitivity and specificity. (Carr-Hill R, et al., J Health Serv Res Policy 1998, 3:207-213; Lipton R B, et al., Rev Contemp Pharmacother 2000,11:63-67; Blau J N, MacGregor E A, Headache 1995, 35:104-106; MacGregor E A, Neurology 1997, 48 (suppl 3): S16-S20; Headache Classification Committee of the International Headache Society, Cephalalgia. 1988, 8 Suppl 7:1-96; Smetana G W, Arch Intern Med 2000, 160:2729-2737)

A large community survey (Lipton R B, et al., Headache, 2001, 41:646-657) found that approximately 75% of migraineurs reported severe disability or complete bed rest as a functional outcome of their acute migraine. Despite this, the extent of disability associated with migraine is not only under-estimated, but is frequently not part of the evaluation of the headache patient, including assessments by headache experts. The lack of physician-patient communication regarding migraine-related disability appears to contribute significantly to the under-diagnosis of the illness. Even when migraine is correctly diagnosed, treatment is frequently not appropriately matched based on disability. The only randomized, double-blind trial designed to evaluate competing migraine treatment strategies found that matching treatment based on disability (“stratified care”) yielded significantly better outcomes then initiating treatment with OTC medication and then moving up to migraine-specific therapy in the event of non-response (“step care”). The public health importance of this finding is underscored by research suggesting that delay in prescribing effective treatment may result in failure to follow up with subsequent medical consultation. The extent to which screening positive on the ID Migraine™ instrument identifies patients who would benefit from migraine-specific treatment with a triptan, as opposed to initial OTC treatment (the step-care strategy) should be tested in a clinical trial.

The ID Migraine™ Screener showed similar sensitivities and specificities across various clinically relevant subgroups (Table 2). Rates of previous migraine diagnosis in this study (28%) were similar to what has been recently reported in the community. (Stewart W F, et al., Cephalalgia 1999,19:107-114) The previously undiagnosed migraine sufferers identified in primary care had moderate-to-high levels of disability, losing an average of 6.2 (±8.6) days from work due to headache over the previous 3 months. These individuals also experienced significant HR-QoL decrements when compared to the general population. In view of this low overall diagnostic recognition rate, an instrument that performs at the level of the ID Migraine™ Screener represents a significant and potentially useful screening tool for primary care healthcare providers and their patients. Given the availability of effective treatment, the use of this screening tool might represent an important step toward reducing the burden of an illness that the WHO has identified, based on a recent global survey, as being one of the top 20 in the world.

Strengths and Limitations of the Current Study

The strengths of the study lie in the large primary care sample and the use of an IHS gold standard diagnosis of migraine by headache specialists. Possible study limitations include uncertainty regarding the representativeness of both the participating PCP practices and the patient sample. Though attrition was relatively low (20%), the decision to participate, and to accept referral to a headache expert, might introduce bias, while study procedures may also, and unavoidably, have influenced results.

Diagnosis of migraine in the primary care setting remains relatively low, despite its high prevalence and associated severity and disability. The results of this study confirm the validity and reliability of a brief, self-administered screening instrument, the 3 item ID Migraine™ Screener, with excellent performance characteristics that recommends it as a simple method for increasing the recognition of migraine in the primary care setting.

TABLE 1
Sensitivity and Specificity of Individual Screener Items vs.
the “Gold Standard” Migraine Diagnosis

| Item number* | Sensitivity | 95% Cl | Specificity | 95% Cl |
|---|---|---|---|---|
| 1. Pain is worse on just | 0.75 | 0.70-0.79 | 0.50 | 0.39-0.61 |
| one side | | | | |
| 2. Pain is pounding, | 0.87 | 0.83-0.90 | 0.22 | 0.14-0.33 |
| pulsing, or throbbing | | | | |
| 3. Pain is moderate | 0.94 | 0.91-0.96 | 0.16 | 0.09-0.25 |
| or severe | | | | |
| 4. Pain is made worse by | 0.67 | 0.62-0.72 | 0.57 | 0.46-0.68 |
| activities such as walking | | | | |
| or climbing stairs | | | | |
| 5. You feel nauseated or | 0.60 | 0.55-0.65 | 0.81 | 0.71-0.89 |
| sick to your stomach | | | | |
| 6. You see spots, stars, | 0.43 | 0.38-0.48 | 0.74 | 0.63-0.83 |
| zigzags, lines or gray | | | | |
| areas for several minutes | | | | |
| or more before or during | | | | |
| your headaches (aura | | | | |
| symptoms) | | | | |
| 7. Light bothers you (a | 0.75 | 0.71-0.80 | 0.74 | 0.63-0.83 |
| lot more than when you | | | | |
| don't have headaches) | | | | |
| 8. Sound bothers you (a | 0.83 | 0.78-0.86 | 0.56 | 0.45-0.67 |
| lot more than when you | | | | |
| don't have headaches) | | | | |
| 9. Functional impairment | 0.87 | 0.83-0.90 | 0.52 | 0.40-0.63 |
| due to headache in last | | | | |
| 3 months† | | | | |

*Ns range from 438 to 448 due to occasional missing values.
†Scored positive if disability reported on any one day in the past 3 months.

TABLE 2
Sensitivity and Specificity of 3-item ID Migraine ™ Screener
in Total Sample and in Clinically Relevant Subgroups

| Item* | Sensitivity | 95% Cl | Specificity | 95% Cl |
|---|---|---|---|---|
| Total validation sample | 0.81 | 0.77-0.85 | 0.75 | 0.64-0.84 |
| (N = 443)* | | | | |
| Male (N = 110) | 0.65 | 0.53-0.76 | 0.80 | 0.63-0.92 |
| Female (N = 333) | 0.86 | 0.81-0.89 | 0.71 | 0.57-0.83 |
| Ages 18-44 (N = 274) | 0.83 | 0.77-0.87 | 0.76 | 0.62-0.87 |
| Ages 45-55 (N = 169) | 0.79 | 0.71-0.86 | 0.74 | 0.56-0.87 |
| No previous diagnosis | 0.76 | 0.70-0.82 | 0.75 | 0.63-0.84 |
| (N = 291) | | | | |
| Previous diagnosis | 0.89 | 0.83-0.94 | 0.77 | 0.46-0.95 |
| (N = 152) | | | | |
| Migraine only (N = 184) | 0.80 | 0.73-0.85 | N/A | N/A |
| Migraine + other | 0.83 | 0.76-0.89 | N/A | N/A |
| headache (N = 148) | | | | |

Validation data was missing on 8 subjects: 1 subject could not be classified based on the physician assessment. An additional 7 subjects were missing one of the key three items from the screener. The resulting sample size available for validation was 443.

What is claimed is:

1. A method of diagnosing migraine in a subject validly and reliably which comprises (a) determining the degree of disability of said subject; (b) determining whether said subject is suffering from nausea; and, (c) determining whether said subject is suffering from sensitivity to light.

2. The method of claim 1, wherein determining said points (a), (b) and (c) comprises asking said subject exactly three questions addressing points (a), (b) and (c), respectively.

3. The method of claim 1, wherein said method has a predictive value of greater than 90 percent.

4. The method of claim 1, wherein said method may be self-administered by said subject or administered by a medical professional.
